(19) United States
(12) Patent Application Publication
(10) Pub. No.: US 2014/0013954 A1
(43) Pub. Date: Jan. 16, 2014

(54) OXYGEN TANK UNIT FOR OXYGEN ENRICHER

(71) Applicant: Michiya Sato, Saitama (JP)

(72) Inventor: Michiya Sato, Saitama (JP)

(73) Assignee: Fujikura Rubber Ltd., Tokyo (JP)

(21) Appl. No.: 14/006,989

(22) PCT Filed: Feb. 16, 2012
(86) PCT No.: PCT/JP2012/053620

§ 371 Date: Sep. 24, 2013

(30) Foreign Application Priority Data
Mar. 25, 2011 (JP) 2011-067250

(51) Int. Cl. A61M 16/10

(52) U.S. Cl. CPC A61M 16/10
USPC 96/113

(57) ABSTRACT

An oxygen-enricher oxygen tank unit includes a single oxygen tank body, to which a pair of nitrogen absorption containers, which alternately receive a supply of compressed air, are connected; a check valve provided between each of the pair of nitrogen absorption containers and the oxygen tank body, wherein each check valve allows gas to flow from an associated nitrogen absorption container to the oxygen tank body, and does not allow gas to flow in a reverse direction thereto; and a pressure-reducing valve, having an oxygen outlet, which is connected to the oxygen tank body. At least one of the pressure-reducing valve and a pair of nitrogen absorption container connector-cylinders, which is provided with the check valve, is directly attached to a body-wall surface of the oxygen tank body. Hence, the structure around the oxygen tank body is simplified and unitized.

### CROSS REFERENCE TO RELATED APPLICATIONS

This application claims the priority of Japanese patent application No. 2011-067250, filed on Mar. 25, 2011 and PCT Application No. PCT/JP2012/053620, filed on Feb. 16, 2012, the disclosures of which are incorporated herein by reference in their entirety.

### TECHNICAL FIELD

The present invention relates to an oxygen tank unit which is used in a medical oxygen enricher.

### BACKGROUND ART

Oxygen enrichers, which generate oxygen by using an absorption material (typically zeolite) which selectively absorbs nitrogen from air, are in practical use as medical oxygen enrichers.

FIG. 10 shows a typical piping system diagram of such an oxygen enricher 10. Compressed air that has been compressed in a compressor 11 is supplied to each inlet of a pair of nitrogen absorption containers 17 and 18 via conduits 12, 13, 14 and pressurized switching valves (electromagnetic ON/OFF valves) 15 and 16. Outlets of the nitrogen absorption containers 17 are connected to a single oxygen tank 24 via conduits 19, 20, 21 and check valves 22 and 23, and the oxygen tank 24 is connected to an air outlet 27 via a conduit 25 and a pressure-reducing valve (regulator) 26. The nitrogen absorption containers 17 and 18 are filled with, e.g., zeolite (powder or granules), which constitutes a nitrogen absorption material which selectively absorbs nitrogen in the air inside the nitrogen absorption containers 17 and 18. The check valves 22 and 23 are one-way valves which allow gas (air) to flow from the nitrogen absorption containers 17 and 18 to the oxygen tank 24 and do not allow gas to flow in the reverse direction.

Conduits 31 and 32 are connected to the conduits 13 and 14 at the downstream sides of the pressurized switching valves 15 and 16 (the inlet sides of the nitrogen absorption containers 17 and 18), respectively, and pressure-reducing switching valves (electromagnetic ON/OFF valves) 33 and 34 are provided on the conduits 31 and 32, respectively. The outlet sides of the pressure-reducing switching valves 33 and 34 are merged at a conduit 35 and are connected to an exhaust muffler 36.

The conduits 19 and 20 are connected to each other via a conduit 37 at the upstream sides of the check valves 22 and 23 (the outlet sides of the nitrogen absorption containers 17 and 18), and a purge valve 40, sandwiched in between throttle valves (orifices) 38 and 39, is provided in the conduit 37.

The ON/OFF control of the pressurized switching valves 15 and 16, the pressure-reducing switching valves 33 and 34, and the purge valve 40 of the oxygen enricher 10 are carried out in accordance with the time chart shown in FIG. 11. Namely, when the pressurized switching valve 15 (16) opens, the pressurized switching valve 16 (15) is closed, and upon a predetermined amount of time lapsing from when the pressurized switching valve 15 (16) opens, the pressure-reducing switching valve 34 (33) opens. Since the pressurized switching valve 16 (15) is closed when the pressurized switching valve 15 (16) opens, pressurized air from the compressor 11 is fed only to the nitrogen absorption container 17 (18), so that nitrogen in the air is absorbed in the absorption material inside the nitrogen absorption container 17 (18) and a high concentration of oxygen is fed through the conduit 19 (20). When the pressure inside the conduit 19 (20) exceeds a predetermined value, the check valve 22 (23) opens so that highly concentrated oxygen is accumulated inside the oxygen tank 24.

On the other hand, upon a predetermined amount of time lapsing after the pressurized switching valve 15 (16) has opened, the pressure-reducing switching valve 34 (33) is opened, and furthermore, upon a predetermined amount of time lapsing after the pressure-reducing switching valve 34 (33) has opened, the purge valve 40 is opened. Consequently, the highly concentrated oxygen on the high-pressure side is supplied to the nitrogen absorption container 18 (17), which was on the low-pressure side, from the upstream side and flows in reverse to the nitrogen absorption container 18 (17). Accordingly, the nitrogen that was absorbed in the absorption material inside the nitrogen absorption container 18 (17) is discharged to the conduit 32 (31) with the highly concentrated air, and the gas containing the discharged nitrogen is discharged via the conduit 35 and the exhaust muffler 36.

The highly concentrated oxygen that has accumulated in the oxygen tank 24 is supplied to the patient from the air outlet 27 after the pressure thereof is reduced by the pressure-reducing valve (regulator) 26. Namely, since the pressure inside the oxygen tank 24 fluctuates greatly as the result of high-pressure highly concentrated oxygen being alternately supplied from the nitrogen absorption containers 17 and 18, highly concentrated oxygen having a reduced pressure fluctuation via the pressure-reducing valve 26 is supplied to the patient. The above description is the operating principle of the oxygen enricher 10.

### SUMMARY OF INVENTION

### Technical Problem

The oxygen enricher 10, which operates according to the above-described operating principle, requires a large number of conduits for connecting the compressor 11, the pressurized switching valves 15 and 16, the pressure-reducing switching valves 33 and 34, the check valves 22 and 23, the purge valve 40, the oxygen tank 24 and the pressure-reducing valve 26, etc., and accordingly, the entire device becomes large, and also incurs an increased assembly cost.

An object of the present invention is to achieve an oxygen tank unit for an oxygen enricher which can simplify and unitize the configuration among the oxygen tank 24, the check valves 22 and 23, and the pressure-reducing valve 26 of the oxygen enricher 10, having the above-described operating principle, with special attention to the configuration around the oxygen tank 24.

### Solution to Problem

The present invention is characterized by an oxygen-enricher oxygen tank unit, including a single oxygen tank body, to which a pair of nitrogen absorption containers, which alternately receive a supply of compressed air, are connected; a check valve provided between each of the pair of nitrogen absorption containers and the oxygen tank body, wherein each check valve allows gas to flow from an associated nitrogen absorption container to the oxygen tank body, and does not allow gas to flow in a reverse direction thereto; and a pressure-reducing valve, having an oxygen outlet, which is connected to the oxygen tank body. At least one of the pressure-reducing valve and a pair of nitrogen absorption container connector-cylinders, which is provided with the check valve, is directly attached to a body-wall surface of the oxygen tank body.

In an embodiment of the present invention, a bacteria filter unit can be connected to an outlet of the pressure-reducing valve of the oxygen tank body.

The oxygen tank body can be further provided with at least one of an oxygen pressure sensor and an oxygen concentration sensor.

It is desirable for the oxygen concentration sensor to be provided at the outlet of the pressure-reducing valve.

Each of the pair of nitrogen absorption container connector-cylinders, provided with the check valve, can, for example, be provided with a check valve unit which is inserted into a stepped through-hole portion in the body-wall surface of the oxygen tank body, and a nitrogen absorption container connector-pipe which is mounted onto the check valve unit in a coaxial manner therewith.

In an embodiment of the present invention, the pressure-reducing valve includes a main housing and a sub-housing which is connected to the main housing. The main housing is mounted on the body-wall surface of the oxygen tank body, and includes a primary pressure-introduction channel which is communicatively connected with the through-hole of the body-wall surface of the oxygen tank body, a secondary pressure-outlet channel, and a main valve which is provided between the primary pressure-introduction channel and the secondary pressure-outlet channel. The sub-housing supports an operational diaphragm assembly between the sub-housing and the main housing, and forms a secondary pressure chamber which is communicatively connected with the secondary pressure-outlet channel. The operational diaphragm assembly and the main valve operate in cooperation with each other so that the main valve opens and closes in accordance with a fluctuation in pressure in the secondary pressure chamber.

A lower housing, of the bacteria filter unit, which is communicatively connected with the secondary pressure-outlet channel, can be mounted onto the main housing of the pressure-reducing valve, and an upper housing, which sandwiches a bacterial filter between the upper housing and the lower housing, can be mounted onto the lower housing.

In an embodiment, the main housing is supported to be detachably attached to the oxygen tank body via bayonet claws.

### Advantageous Effects of Invention

According to the present invention, in an oxygen enricher which utilizes a pair of nitrogen absorption containers, since at least one of the pressure-reducing valve and the pair of nitrogen absorption container connector-cylinders, which are provided with a check valve, are directly attached to a body-wall surface of a oxygen tank, the configuration among the oxygen tank body, the check valve and the pressure-reducing valve can be simplified and unitized.

### BRIEF DESCRIPTION OF DRAWINGS

FIG. 1 is a perspective view showing an embodiment of an oxygen tank unit, according to the present invention, which is utilized in an oxygen enricher.

FIG. 2 is a side elevation of the embodiment of an oxygen tank unit.

FIG. 3 is a sectional view taken along the line III-III shown in FIG. 2.

FIG. 4 is an enlarged view of the IV-section shown in FIG. 3.

FIG. 5 is a perspective view of the section in FIG. 4.

FIG. 6 is an enlarged view of the IV-section shown in FIG. 3.

FIG. 7 is a perspective view of the section in FIG. 6.

FIG. 8 is a circuit diagram of the oxygen tank unit of the present invention.

FIG. 9 is a perspective view of the fundamental components, showing another embodiment of an oxygen tank unit, according to the present invention.

FIG. 10 is a circuit diagram of an oxygen enricher which constitutes a premise to the present invention.

FIG. 11 is a timing chart showing the ON/OFF timing of each valve of the oxygen enricher of FIG. 10.

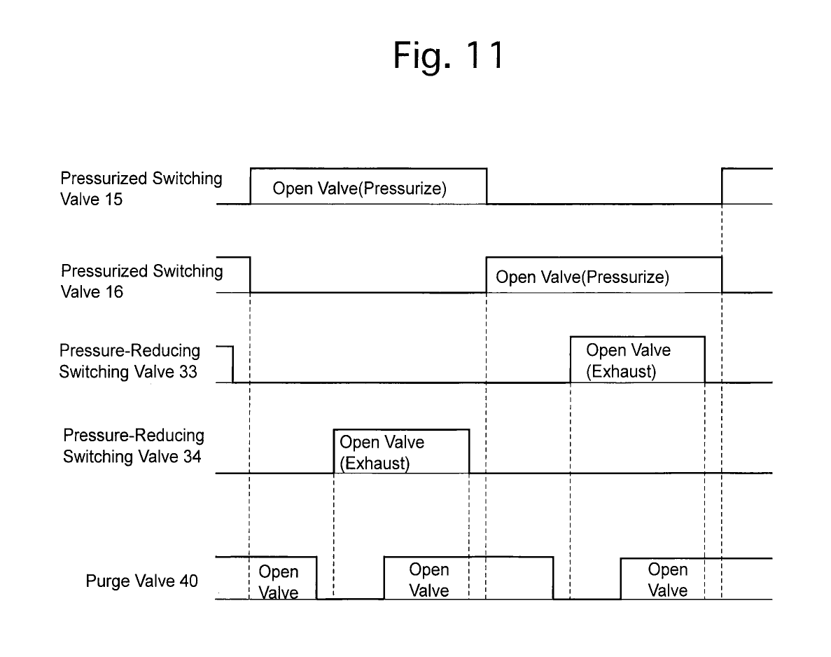

### DESCRIPTION OF THE PREFERRED EMBODIMENTS

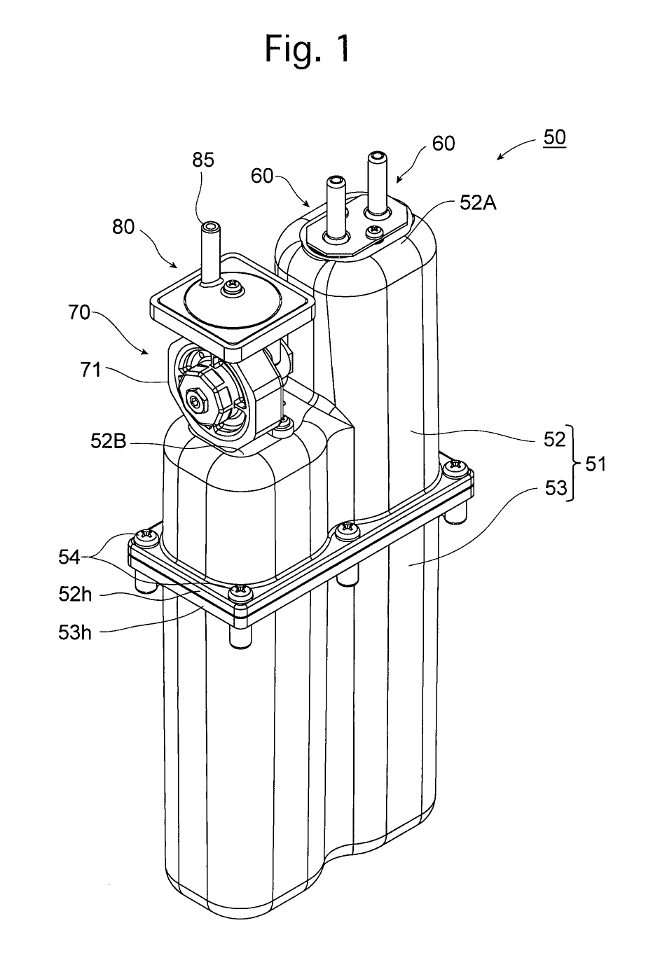

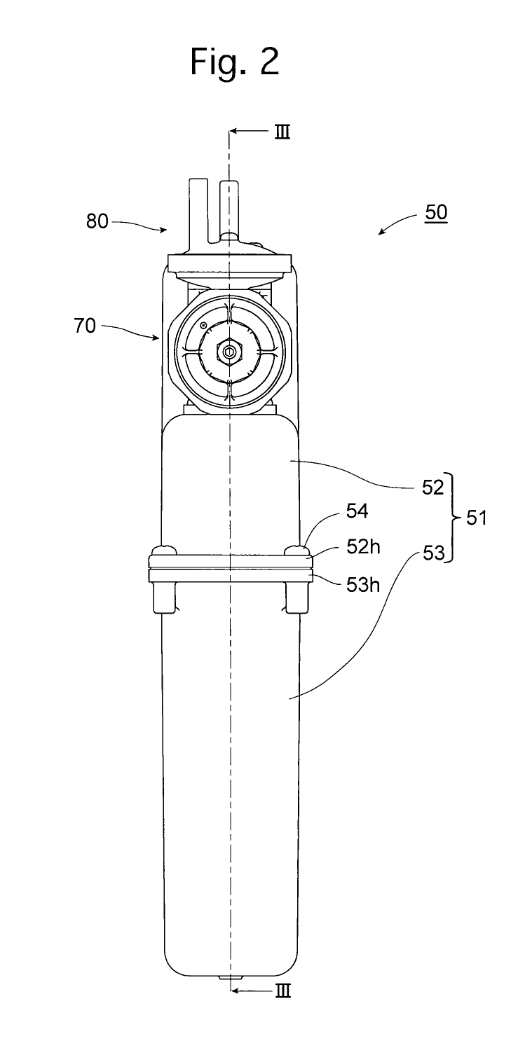

FIGS. 1 through 8 show a first embodiment of an oxygen-enricher oxygen tank unit 50 according to the present invention. As shown in FIGS. 1 through 3, the oxygen-enricher oxygen tank unit 50 includes a compound-resin oxygen tank body 51. The oxygen tank body 51 includes half bodies 52 and 53, and respective flanges 52h and 53h are joined together with fixing bolts 54 to form an air-tight space therein.

The tank half body 52 includes a high end-wall 52A and a low end-wall 52B which are mutually parallel to each other and have different heights. A pair of in-built check-valve cylinders (nitrogen absorption container connector-cylinders) 60 are mounted onto the high end-wall 52A so as to be orthogonal to the high end-wall 52A, and a pressure-reducing valve (regulator) 70 is mounted onto the low end-wall 52B.

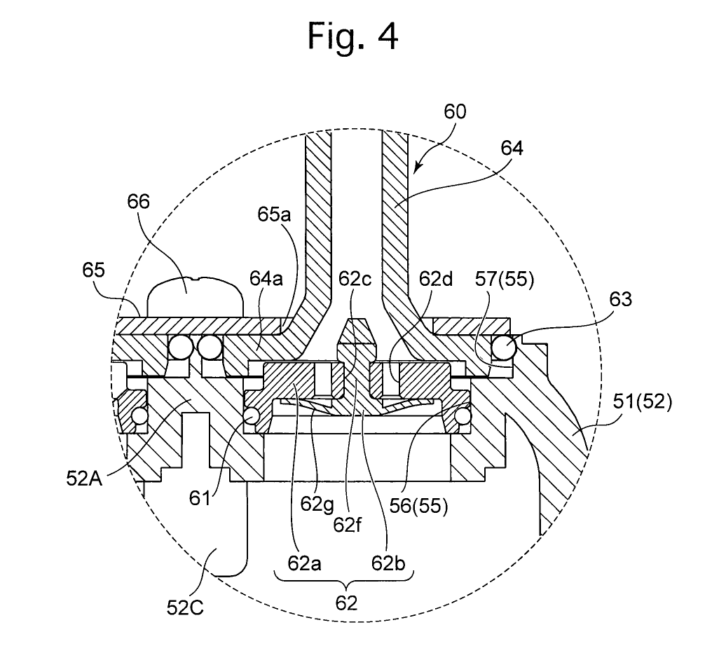

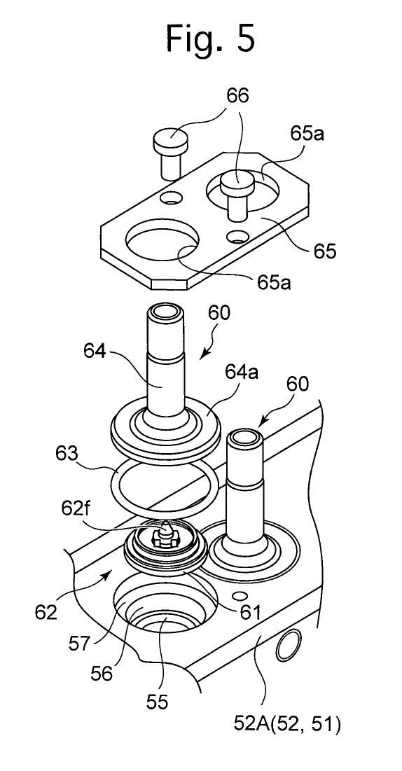

FIGS. 4 and 5 show the detailed structure of the in-built check-valve cylinders (nitrogen absorption container connector-cylinders) 60. A pair of stepped through-holes 55, which correspond to the pair of in-built check-valve cylinders 60, are formed in the high end-wall 52A. Each of these stepped through-holes 55 includes a small-diameter stepped portion 56 and a large-diameter stepped portion 57. Each in-built check-valve cylinder 60 includes a check valve unit 62 which is air-tightly inserted into the small-diameter stepped portion 56 of the stepped through-holes 55 via an O-ring 61, and a nitrogen absorption container connector-pipe 64 which is air-tightly inserted into the large-diameter stepped portion 57 via an O-ring 63. The check valve unit 62 is configured of a flat circular valve-seat 62a and a valve body 62b. A valve-body holding hole 62c is formed in the flat circular valve-seat 62a, at the central portion thereof, and a plurality of through-holes 62d are formed in a surrounding portion of the flat circular valve-seat 62a. The valve body 62b includes a shaft portion 62f which is insertably held in the valve-body holding hole 62c, and a valve portion 62g which normally closes over the through-holes 62d. The valve portion 62g of the valve body 62b closes the through-holes 62d by the pressure inside the oxygen tank body 51, and the valve body 62b is installed in the valve-body holding hole 62c so as to deform in a direction to open the through-holes 62d by pressure outside the oxygen tank body 51. A large-diameter flange 64a, which is inserted into the large-diameter stepped portion 57, is formed on the bottom end of the nitrogen absorption container connector-pipe 64.

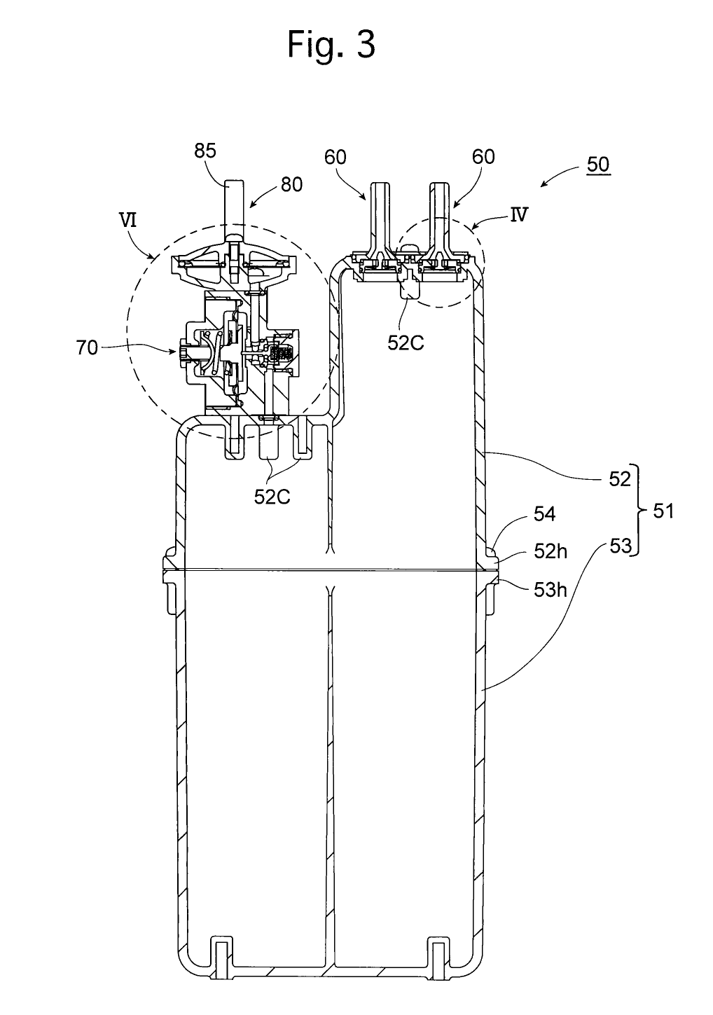

The pair of in-built check-valve cylinders 60 have the same structure, and are fixed to the high end-wall 52A of the tank half body 52 by a single mounting plate 65. Namely, a pair of through-holes 65a corresponding to the pair of nitrogen absorption container connector-pipes 64 is formed in the mounting plate 65, and the mounting plate 65 is mounted onto the high end-wall 52A by mounting screws 66 with the pair of nitrogen absorption container connector-pipes 64 inserted into the pair of through-holes 65a. The mounting plate 65 mounts, while pressing, the check valve units 62 into the small-diameter stepped portions 56 of the stepped through-holes 55, via the large-diameter flanges 64a. Screw-bearing seats 52C (FIGS. 3 and 4), into which the mounting screws 66 fixedly screw-engage, are formed on the inner surface of the high end-wall 52A of the tank half body 52.

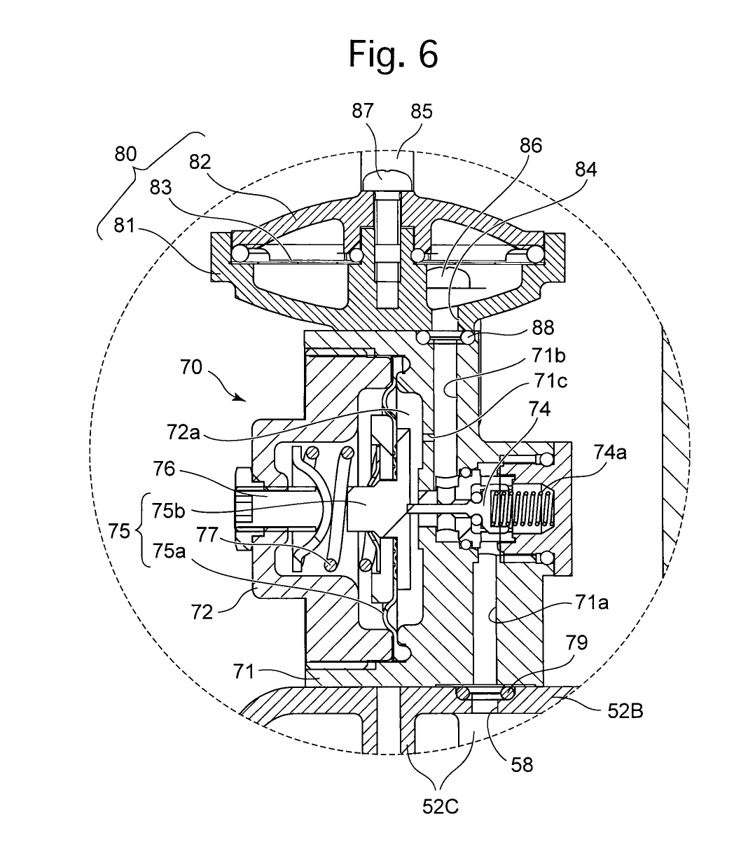

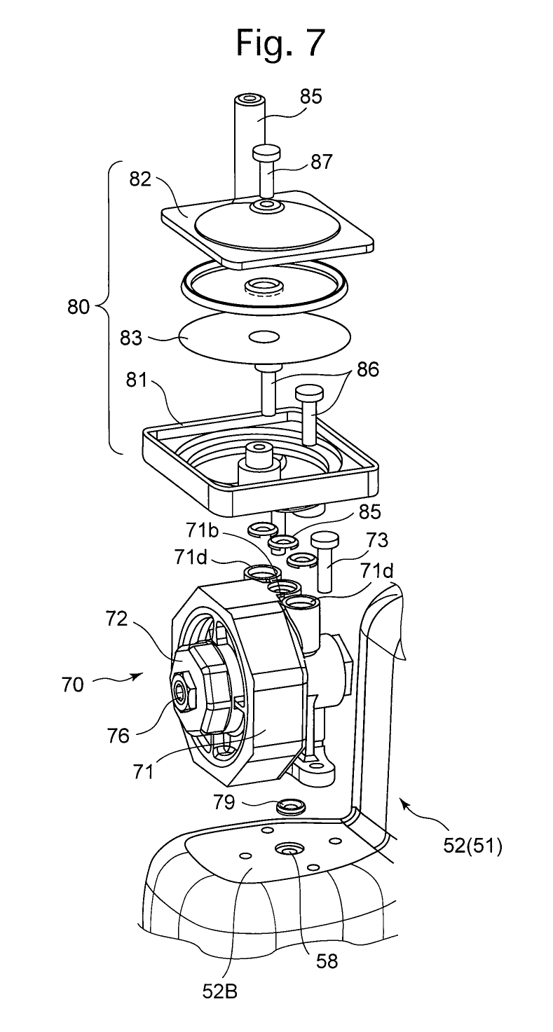

FIGS. 6 and 7 show a detailed structure of the pressure-reducing valve 70. A through-hole 58 is formed in the low end-wall 52B of the tank half body 52. The pressure-reducing valve 70 includes a main housing 71 and a sub-housing 72. The main housing 71 is mounted to the low end-wall 52B via mounting screws 73 (FIG. 7). Other screw-bearing seats 52C (FIGS. 3 and 6), into which the mounting screws 73 fixedly screw-engage, are formed on the inner surface of the low end-wall 52B.

The main housing 71 includes a primary pressure-introduction channel 71a, which is directly communicatively-connected to the through-hole 58 via an O-ring 79, and a secondary pressure-outlet channel (oxygen outlet) 71b. A main valve 74 is provided in a communicative-connection channel that communicatively connects the primary pressure-introduction channel 71a with the secondary pressure-outlet channel 71b. The main valve 74 is a valve that normally shuts-off the communicative connection between the primary pressure-introduction channel 71a and the secondary pressure-outlet channel 71b by a valve-closing spring 74a.

The sub-housing 72 defines a secondary pressure chamber 72a which sandwiches an operational diaphragm assembly 75 between the sub-housing 72 and the main housing 71. The secondary pressure chamber 72a is communicatively connected with the secondary pressure-outlet channel 71b via a connection channel 71c. The operational diaphragm assembly 75 includes a diaphragm 75a, and an operational piston 75b that is mounted at a central portion of the diaphragm 75a. The operational piston 75b opens and closes in cooperation with the main valve 74 in accordance with the fluctuation in pressure of the secondary pressure chamber 72a (secondary pressure-outlet channel 71b). Specifically, when the pressure inside the secondary pressure-outlet channel 71b lowers, the operational diaphragm assembly 75 moves the main valve 74 in the valve-closing direction against the force of the valve-closing spring 74a, and when the pressure inside the secondary pressure-outlet channel 71b increases, the operational diaphragm assembly 75 conversely moves away from the main valve 74 to thereby close the main valve 74. As a result of this operation being repeated in accordance with the pressure fluctuation of the secondary pressure-outlet channel 71b, the pressure discharging from the secondary pressure-outlet channel 71b is maintained substantially constant. The discharge pressure of the secondary pressure-outlet channel 71b can be adjusted by adjusting the force of a pressure-adjustment spring 77, which is exerted on the operational diaphragm assembly 75, by rotating a pressure-adjustment screw 76.

A bacteria filter unit 80, which is communicatively connected to the secondary pressure-outlet channel 71b, is mounted on the main housing 71 of the pressure-reducing valve 70. The bacteria filter unit 80 supports, in a sandwiched manner, a bacterial filter 83 in between a lower housing 81 and an upper housing 82. A gas inlet 84 which is communicatively connected with the secondary pressure-outlet channel 71b is formed in the insulator 81, and a gas discharge outlet (oxygen outlet) 85 for discharging gas (oxygen) passing through the bacterial filter 83 is mounted on the upper housing 82. The lower housing 81 of the bacteria filter unit 80 is mounted onto the main housing 71 of the pressure-reducing valve 70 via mounting screws 86 while maintaining an air-tight state and sandwiching an O-ring 88 between the inlet of the gas inlet 84 and the outlet of the secondary pressure-outlet channel 71b. The upper housing 82 is mounted onto the lower housing 81, which is mounted onto the main housing 71, by mounting screws 87 to sandwich the bacterial filter 83 therebetween. Screw seats 71d (FIG. 7), into which the mounting screws 86 are screw-engaged, are formed in the main housing 71. The bacterial filter 83 is a commonly known filter which removes impurities such as bacteria that are contained in the oxygen that passes therethrough. The bacterial filter 83 is replaced after being in use for a predetermined amount of time.

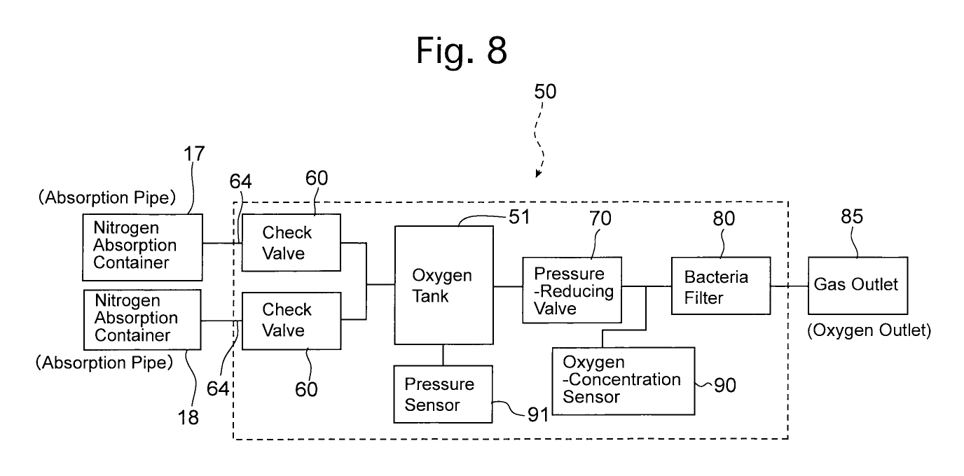

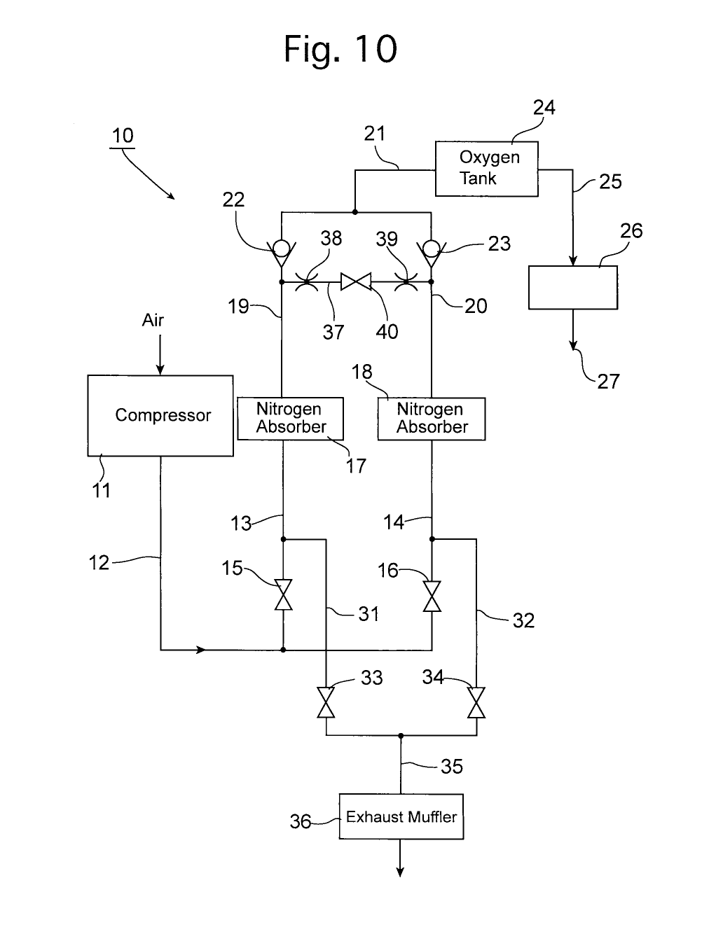

FIG. 8 is a circuit diagram of the oxygen-enricher oxygen tank unit 50 in which the in-built check-valve cylinders 60 and the pressure-reducing valve 70 (and the bacteria filter unit 80) are mounted onto the high end-wall 52A and the low end-wall 52B, respectively. The pair of nitrogen absorption container connector-pipes 64 are connected to the nitrogen absorption containers 17 and 18, which are described in FIG. 10, by a suitable conduit means, and the oxygen from the gas discharge outlet 85 is given to the user's (patient's) mouth (nose) via a soft supply tube or aspirator. As shown in FIG. 8, it is desirable for an oxygen concentration sensor 90 to be provided downstream from the outlet of the pressure-reducing valve 70, and for an oxygen pressure sensor 91 to be provided in the oxygen tank body 51. The output of these sensors is input into a control circuit.

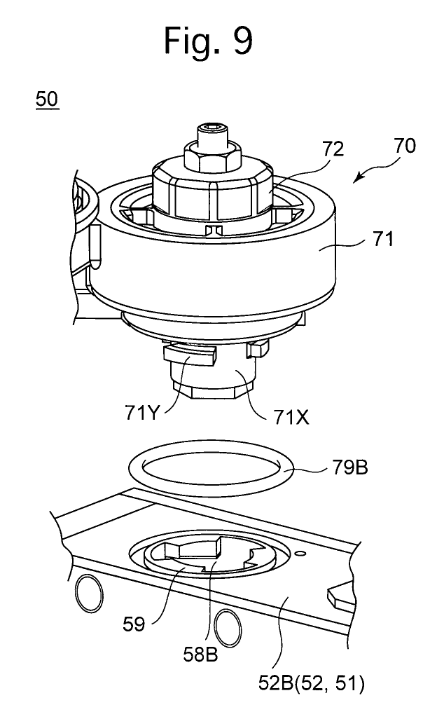

FIG. 9 shows another embodiment of the oxygen tank unit 50 according to the present invention. This embodiment shows a bayonet-type pressure-reducing valve 70, to which the low end-wall 52B of the tank half body 52 is installed. A plurality of bayonet claws 59 are formed on the inner peripheral portion of the large-diameter through-hole 58B, which is formed in the low end-wall 52B, so as to project from the inner peripheral portion of the large-diameter through-hole 58. A cylindrical portion 71X, which fits into the through-hole 58B, and bayonet claws 71Y, which are detachably connected with the bayonet claws 59, are formed on the main housing 71 of the pressure-reducing valve 70. The bayonet claws 59 and the bayonet claws 71Y connect/detach by relatively rotating the cylindrical portion 71X with the cylindrical portion 71X inserted into the through-hole 58B, in a manner similar to that of well known in interchangeable lenses for SLR cameras, etc. The primary pressure-introduction channel 71a, corresponding to the primary pressure-introduction channel 71a of FIG. 6, that is communicatively connected with the through-hole 58B is open at the cylindrical portion 71X. The primary pressure-introduction channel and the through-hole 58B are connected to each other in an air-tight manner via a large-diameter O-ring 79B. The fundamental structure inside the pressure-reducing valve 70 is the same as the structure of the pressure-reducing valve 70 of FIG. 6.

In the above embodiments, a pair of in-built check-valve cylinders (nitrogen absorption container connector-cylinders) 60 and a pressure-reducing valve 70 are both directly attached to the oxygen tank body 51, however, a predetermined simplification in structure can be achieved even with only one thereof being directly attached to the oxygen tank body 51. Furthermore, in the above embodiments, although the bacteria filter unit 80 is mounted onto the main housing 71 of the pressure-reducing valve 70, an embodiment (in which the secondary pressure-outlet channel 71b of the pressure-reducing valve 70 is a direct air outlet) is also possible in which the bacteria filter unit 80 is omitted.

### INDUSTRIAL APPLICABILITY

The oxygen-enricher oxygen tank unit of the present invention can be widely utilized for medical purposes.

### REFERENCE SIGNS LIST

10 Oxygen enricher
11 Compressor
12 13 14 19 20 21 25 31 32 35 Conduits
15 16 Pressurized switching valves
17 18 Nitrogen absorption containers
22 23 Check valves
24 Oxygen tank
26 Pressure-reducing valve
33 34 Pressure-reducing switching valves
36 Exhaust muffler
37 Conduit
38 39 Throttle valves (orifices)
40 Purge valve
50 Oxygen-enricher oxygen tank unit
51 Oxygen tank body
52 53 Tank half body
52A High end-wall
52B Low end-wall
52C Screw-bearing seats
54 Fixing bolts
55 Stepped through-holes
56 Small-diameter stepped portion
57 Large-diameter stepped portion
58 58B Through-hole
59 Bayonet claws
60 In-built check-valve cylinders (nitrogen absorption container connector-cylinders)
62 Check valve unit
62a Valve-seat
62b Valve body
62c Valve-body holding hole
62d Through-holes
63 O-ring
64 Nitrogen absorption container connector-pipe
65 Mounting plate
65a Through-holes
66 Mounting screws
70 Pressure-reducing valve
71 Main housing
71a Primary pressure-introduction channel
71b Secondary pressure-outlet channel (oxygen outlet)
71d Screw seat
71X Cylindrical portion
71Y Bayonet claws
72 Sub-housing
72a Secondary pressure chamber
73 Mounting screws
74 Main valve
75 Operational diaphragm assembly
80 Bacteria filter unit
81 Lower housing
82 Upper housing
83 Bacterial filter
84 Gas inlet
85 Gas discharge outlet (oxygen outlet)
90 Oxygen concentration sensor
91 Oxygen pressure sensor

1-8. (canceled)

9. An oxygen-enricher oxygen tank unit, comprising:
a single oxygen tank body;
a pair of nitrogen absorption containers, which alternately receive a supply of compressed air, which are formed separately from said tank body;
a check valve provided between each of said pair of nitrogen absorption containers and said oxygen tank body, wherein each said check valve allows gas to flow from an associated nitrogen absorption container to said oxygen tank body, and does not allow gas to flow in a reverse direction thereto;
a pressure-reducing valve, having an oxygen outlet, which is connected to said oxygen tank body; and
a pair of absorption container connection cylinders, which are provided with said check valve, respectively, for connecting said oxygen tank body to said pair of nitrogen absorption containers;
wherein both a pair of said absorption container connection cylinders and said pressure-reducing valve are directly attached to a body-wall surface of said oxygen tank body.

10. The oxygen-enricher oxygen tank unit according to claim 9, wherein a bacteria filter unit is further provided by being connected to an outlet of said pressure-reducing valve of said oxygen tank body.

11. The oxygen-enricher oxygen tank unit according to claim 9, wherein said oxygen tank body is further provided with at least one of an oxygen pressure sensor and an oxygen concentration sensor.

12. The oxygen-enricher oxygen tank unit according to claim 11, wherein said oxygen concentration sensor is provided at said outlet of said pressure-reducing valve.

13. The oxygen-enricher oxygen tank unit according to claim 9, wherein each of said pair of nitrogen absorption container connector-cylinders, provided with said check valve, is provided with a check valve unit which is inserted into a stepped through-hole portion in said body-wall surface of said oxygen tank body, and a nitrogen absorption container connector-pipe which is mounted onto said check valve unit in a coaxial manner therewith.

14. The oxygen-enricher oxygen tank unit according to claim 9, wherein said pressure-reducing valve comprises a main housing and a sub-housing which is connected to said main housing,
wherein said main housing is mounted on said body-wall surface of said oxygen tank body, and includes a primary pressure-introduction channel which is communicatively connected with said through-hole of said body-wall surface of said oxygen tank body, a secondary pressure-outlet channel, and a main valve which is provided between said primary pressure-introduction channel and said secondary pressure-outlet channel;
wherein said sub-housing supports an operational diaphragm assembly between said sub-housing and said main housing, and forms a secondary pressure chamber which is communicatively connected with said secondary pressure-outlet channel, and
wherein said operational diaphragm assembly and said main valve operate in cooperation with each other so that the main valve opens and closes in accordance with a fluctuation in pressure in said secondary pressure chamber.

15. The oxygen-enricher oxygen tank unit according to claim 14, wherein a lower housing, of said bacteria filter unit, which is communicatively connected with said secondary pressure-outlet channel, is mounted onto said main housing, and
wherein an upper housing, which sandwiches a bacterial filter between the upper housing and said lower housing, is mounted onto said lower housing.

16. The oxygen-enricher oxygen tank unit according to claim 14, wherein said main housing is supported to be detachably attached to said oxygen tank body via bayonet claws.
